(19) United States
(12) Patent Application Publication
(10) Pub. No.: US 2014/0187934 A1
(43) Pub. Date: Jul. 3, 2014

(54) SYSTEMS AND METHODS FOR CONFIGURING A MEDICAL DEVICE

(71) Applicant: GENERAL ELECTRIC COMPANY, Schenectady, NY (US)

(72) Inventor: Mark Steven Urness, Wauwatosa, WI (US)

(73) Assignee: General Electric Company, Schenectady, NY (US)

(21) Appl. No.: 13/732,008

(22) Filed: Dec. 31, 2012

(51) Int. Cl. G06Q 10/10; G06Q 50/22; A61B 8/00

(52) U.S. Cl. CPC G06Q 10/10; A61B 8/461; G06Q 50/22
USPC 600/437; 705/2

(57) ABSTRACT

Systems and methods for configuring a medical device are provided. One system includes a computer configured to access a plurality of stored Digital Imaging and Communications in Medicine (DICOM) worklists and transmit DICOM worklist information. The system also includes a medical device configured to receive the DICOM worklist information including examination or scan info nation for the medical device and determine a preset configuration for the medical device corresponding to an examination or scan using the examination or scan information. The medical device is further configured to automatically select the preset configuration to set one or more acquisition parameters for the examination or scan.

### BACKGROUND

The subject matter disclosed herein relates generally to medical imaging systems, and more particularly to setting parameters or protocols for ultrasound imaging systems.

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. The workstation or device may show the ultrasound images through a display device.

Before each imaging session, an operator typically sets up the ultrasound system for the particular type of scan to be performed. In a typical process, an operator accesses a Digital Imaging and Communications in Medicine (DICOM) worklist to select a patient for the upcoming ultrasound scan to be performed. The selection of the patient from the DICOM worklist typically populates the data fields on the screen of the ultrasound system with patient demographic information. Thus, in conventional systems, patient information is loaded into data fields upon selecting the patient from the DICOM worklist. However, although the patient data may be populated in the data fields, an operator still has to configure the device with the scanning parameters or presets for the exam protocol. For example, an operator selects the proper probe and probe settings (e.g., scan settings) for the scan to be performed on the patient.

Thus, while patient demographic information may be loaded based on the selection of the patient from the DICOM worklist, a user must still prepare the ultrasound system for the scan, including manually configuring the scanning settings or presets. In clinical settings an ultrasound system may be shared by multiple departments or an emergency room, which may have to perform multiple different exams. As a result, there is a risk of performing an exam on a mis-configured ultrasound system with conventional set up procedures, which can lead to improper image acquisition and diagnosis. Moreover, different department, clinics or medical facilities may have different workflows associated with the ultrasound system, which can lead to the possibility for additional confusion in setting up the different ultrasound systems, which can also result in improper image acquisition and diagnosis.

### BRIEF DESCRIPTION

In one embodiment, a medical system is provided that includes a computer configured to access a plurality of stored Digital Imaging and Communications in Medicine (DICOM) worklists and transmit DICOM worklist information. The medical system also includes a medical device configured to receive the DICOM worklist information including examination or scan information for the medical device and determine a preset configuration for the medical device corresponding to an examination or scan using the examination or scan information. The medical device is further configured to automatically select the preset configuration to set one or more acquisition parameters for the examination or scan.

In another embodiment, an ultrasound imaging system is provided that includes a user interface having a display configured to display patient and examination information and an examination determination module configured to determine, from received Digital Imaging and Communications in Medicine (DICOM) worklist information including examination or scan information, a preset configuration corresponding to an examination or scan using the examination or scan information. The ultrasound imaging system also includes a presets configuration module configured to automatically select the preset configuration to set one or more acquisition parameters for the examination or scan.

In yet another embodiment, a method for configuring a medical device is provided. The method includes determining, from received Digital. Imaging and Communications in Medicine (DICOM) worklist information including examination or scan information, a preset configuration corresponding to an examination or scan using the examination or scan information. The method also includes automatically selecting the preset configuration to set one or more acquisition parameters for the examination or scan.

### BRIEF DESCRIPTION OF THE DRAWINGS

FIG. 1 is a diagram illustrating a medical setting in which various embodiments may be implemented.

FIG. 2 illustrates a screenshot of a user interface in accordance with an embodiment.

FIG. 3 illustrates a table in accordance with an embodiment.

FIG. 4 illustrates a screenshot of the user interface of FIG. 2 with populated data fields.

FIG. 5 illustrates a screenshot of a user interface in accordance with another embodiment.

FIG. 6 is a flowchart of a method for configuring a medical device in accordance with various embodiments.

FIG. 7 is a block diagram of an ultrasound imaging system in accordance with an embodiment.

FIG. 8 illustrates an ultrasound system in which various embodiments may be implemented.

FIG. 9 illustrates a portable ultrasound imaging system in which various embodiments may be implemented.

FIG. 10 illustrates a console-type ultrasound imaging system in which various embodiments may be implemented.

### DETAILED DESCRIPTION

Embodiments described herein include systems, methods, and computer readable media that may provide an improved set up for configuring one or more medical devices for performing scanning operations. For example, embodiments described herein may automatically upload or select a present configuration for scanning parameters and/or a protocol, which may be determined based on data within a Digital Imaging and Communications in Medicine (DICOM) worklist. In various embodiments, an automatic ultrasound preset configuration is provided using the DICOM worklist. In some embodiments, based on identified data received in DICOM tags, one or more medical devices may be configured with one or more presets (e.g., predetermined settings) for the examination or scan.

A technical effect of at least one embodiment is a safer and/or more effective imaging environment with reduced risk of performing an exam on a mis-configured medical device. At technical effect of at least one embodiment is configuring a medical device automatically that allows the device to be shared at multiple locations and for multiple scans or examinations. A technical effect of at least one embodiment is a more efficient medical examination workflow.

It should be noted that although various embodiments may be described in connection with a particular imaging device or modality, for example, an ultrasound imaging system, the various embodiments may be implemented in other types of medical settings and for different devices or modalities. Also, as used herein in various embodiments, DICOM worklist generally refers to a list of examinations for one or more devices and associated information that may be communicated using the DICOM standard. The DICOM worklist may include, for example, a list of patients (including demographic information) and the corresponding type of scan or examination to be performed for one or more medical devices.

The following detailed description of various embodiments will be better understood when read in conjunction with the appended drawings. To the extent that the figures illustrate diagrams of the functional blocks of the various embodiments, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

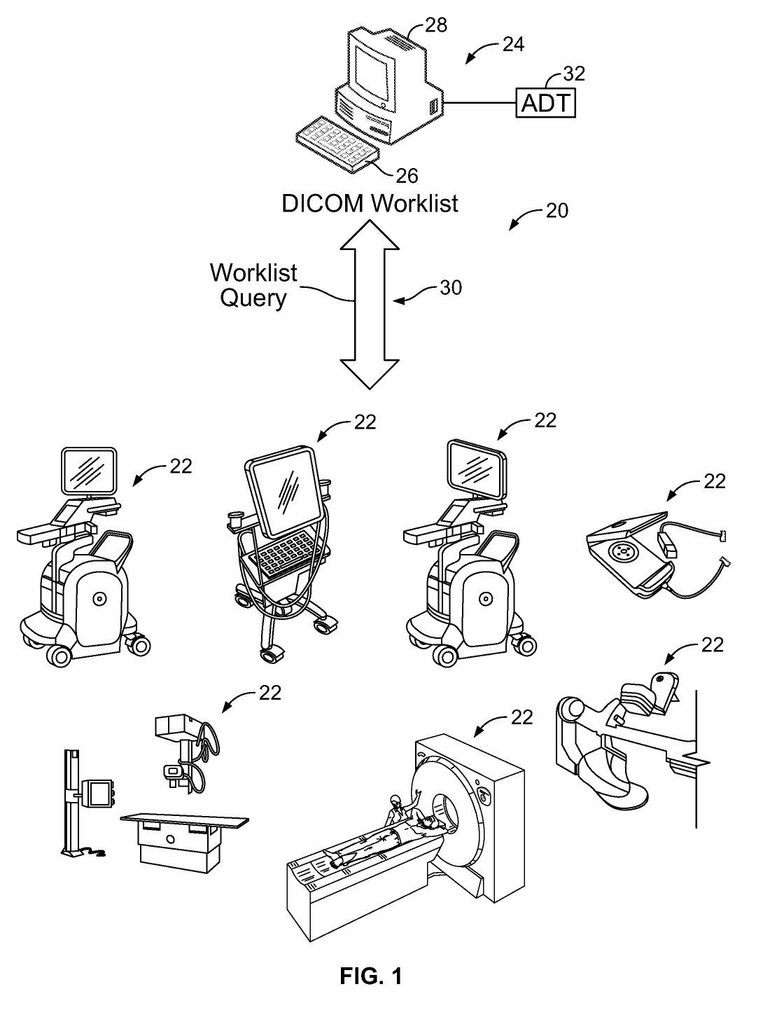

FIG. 1 illustrates a medical setting 20 in which various embodiments may be implemented. The medical setting 20 may correspond to multiples departments within a medical facility or multiple locations at different medical facilities. In the illustrated embodiment, a plurality of medical devices 22 are operable to perform one or more medical examinations or scans. For example, the medical devices 22 may include ultrasound systems or devices, nuclear medicine imaging devices (e.g., Positron Emission Tomography (PET) or Single Photon Emission Computed Tomography (SPECT) imaging systems), Magnetic Resonance (MR) imaging devices, Computed Tomography (CT) imaging devices, and/or x-ray imaging devices, among others. It should be noted that although a description of the operation of an ultrasound system in accordance with various embodiments is provided herein, the various embodiments may be implemented in connection with different ones of the medical devices 22 or other medical devices.

The medical devices are communicatively coupled to a computer 24, which may be a stand-alone computing device, server or other processing machine that accesses a database having stored DICOM worklists. It should be noted that the entry and storage of the DICOM worklists, as well as the access to the stored DICOM worklists may be provided using any suitable DICOM procedure. In various embodiments, the computer 24 includes a user input 26 (e.g., keyboard or mouse) and a display 28 and with which a user or operator may interact. It should be noted that in various embodiments the operator or user is located at the medical device 22 and interacting remotely with the computer 24.

The medical devices 22 and computer 24 communicate over a communication link 30, which may be any suitable wired or wireless connection. For example, the various components may be connected in a local area network (LAN) or similar type of arrangement. Additionally, the medical devices 22 may be coupled to the computer 24 through the same or different communication links 30, which may use the same or different communication protocols for transferring data therebetween.

In various embodiments, the DICOM worklists are generated from information input to an Admissions/Discharge/Transfer (ADT) system 32. For example, information input into the ADT system, such as patient information and scheduling of examinations or scans is used to generate the DICOM worklists. The DICOM worklists may include the date, time, name, patient ID and other information that is acquired from the ADT system. Additionally, the type of examination or scan to be performed also is acquired in various embodiments (e.g., cardiac ultrasound scan, stress echo study or emergency department exam). Thus, in various embodiments the DICOM worklists include information that may be communicated to the medical devices 22 to allow a determination of the patient and type of examination or scan to be performed by the medical device 22. It should be noted that in some embodiments, the ADT system may be coupled or in communication with an Electronic Medical Records (EMR) system that also may provide patient and related information, such as archived patient records. The EMR may, for example, form part of a local stand-alone health information system that allows storage, retrieval and modification of medical records.

In various embodiments, one or more DICOM worklists are communicated from the computer 24 to one or more of the medical devices 22. In various embodiments, the DICOM worklists include patient information (e.g., used to identify the patient) and a description of the examination, scan or study to be performed using the particular medical device 22. Accordingly, in various embodiments, a different DICOM worklist corresponds to each of the medical devices 22.

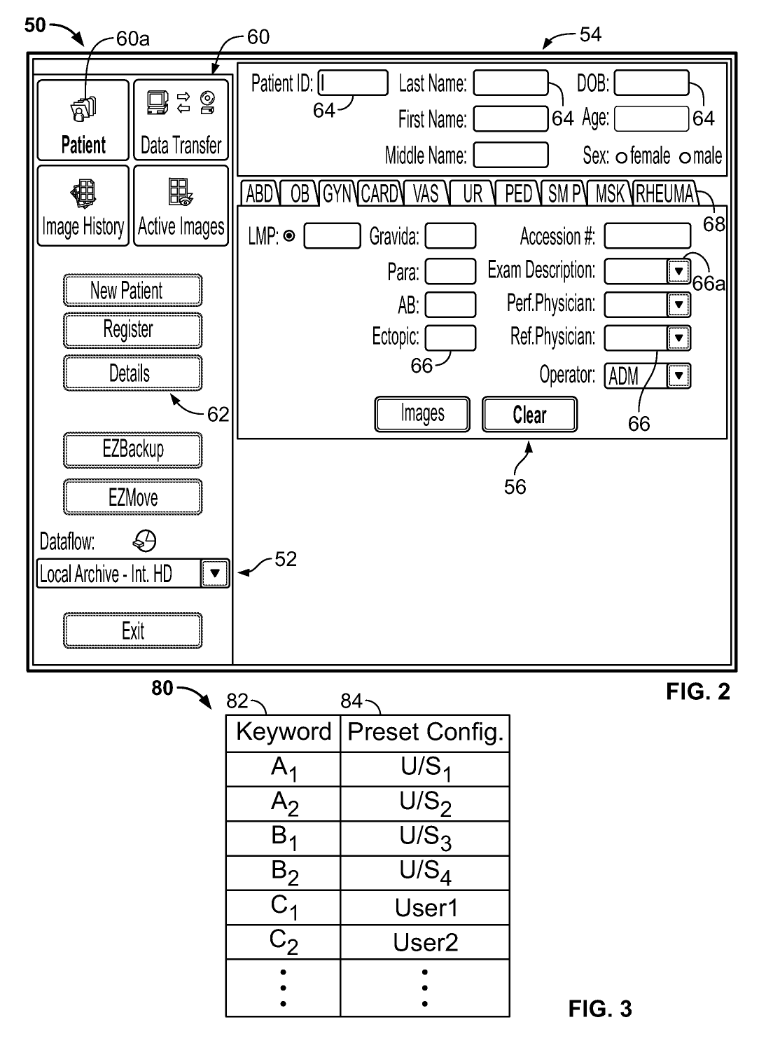

Each of the medical devices 22 includes a user interface that allows a user or operator to interface with the medical device 22. For example, the user interface allows a user to query a DICOM Worklist broker (or other client), such as on the computer 24. In various embodiments, the medical device 22 accesses a DICOM worklist service available on the computer 24, which may be a server or computer system. For example, FIG. 2 illustrates a screenshot of a patient screen 50 that may be displayed on one of the medical devices 22, which in the illustrated embodiment is an ultrasound system. The patient screen 50 may define a user interface that includes user selectable elements to allow a user to interact with the DICOM Worklist broker.

In particular, using the interface, such as via the patient screen 50, a query may be initiated of the DICOM Worklist broker to identify a DICOM Worklist for the medical device 22 or for a particular patient. For example, using the patient screen 50 a query may be initiated for all patients having scheduled examination or scans on a particular date for the medical device 22 or for more than one medical device 22. Based on the worklist query, DICOM Worklist query results are generated by the DICOM Worklist broker. For example, a DICOM Worklist database may be searched to identify search results that satisfy the worklist query. It should be noted that the search may be performed using any suitable search method.

In various embodiments, the search results may include the list of patients with scheduled examinations or scans for a particular day and the medical device to perform the examination (e.g., identified by device number, such as cardiac 01 or emergency room 01). A user may then select the patient for an upcoming examination or scan (e.g., the next patient to examine or scan with the medical device) from the DICOM Worklist, which causes patient data and examination or scan description information to be communicated from the DICOM Worklist broker to the medical device 22. In various embodiments, and using DICOM transfer technology, the demographic data for the selected patient is transmitted and automatically populates corresponding data fields (e.g., name, date of birth, sex and patient ID) on the patient screen 50.

More particularly, the patient screen 50 shown in FIG. 2 illustrates empty data fields before the patient demographic data is automatically populated. In the illustrated embodiment, the patient screen 50 includes a control portion 52, a patient information portion 54 and an examination portion 56. The control portion 52 generally includes user selectable elements (e.g., selectable by a computer mouse or by touch on a touch-screen) that allow navigation through different screens of the user interface. For example, a plurality of user selectable buttons 60 (e.g., virtual displayed buttons) may be provided to select a patient, transfer data to an external device, and view active or archived images. Other selectable buttons 60 may be provided, for example, based on the type of medical device 22 and available options. In the illustrated embodiment, the Patient button 60a is selected, which cause additional buttons 62 to be displayed for controlling different patient selection and entry functions, for example, selecting or registering a new patient by searching for a DICOM Worklist.

In the displayed patient screen, a plurality of patient information fields 64 are provided in the patient information portion 54 of the user interface. By entering search criteria (e.g., patient's last name), such as in one of the fields 64 (or in a different search screen), a DICOM query is initiated, such as a worklist query via the DICOM Worklist broker as described in more detail herein. Additionally, as can be seen, the examination portion 56 includes a plurality of fields that define or describe the examination or scan to be performed by the medical device 22 for the patient selected. It should be noted that the examination portion 56 may include a plurality of tabs 68 corresponding to different examinations or scans. For example, the fields 66 corresponding to each of the examinations or scans (ten selectable scans are illustrated) include an exam description field 66a that defines the examination or scan to be performed.

In various embodiments, as described in more detail herein, the study or exam description is used to automatically configure the medical device 22, which in various embodiments includes automatically selecting one of a plurality of preset configurations using information based on the DICOM Worklist. For example, in some embodiments a plurality of system presets may be defined based on the type of study or examination to be performed. Additionally, in various embodiments, a plurality of user defined preset may be provided. The presets define or set the scanning or imaging parameters (e.g., depth of scanning, gain, focal zone, etc.). Thus, the scanning parameters for a particular type of scan are associated with presets that may be determined based on the type of examination or scan, as well as user defined settings. For example, in one embodiment, for an ultrasound scan, preset/probe settings (e.g., MSK, RHEUMA, Vascular, etc.) are automatically selected based on the examination or scan description that forms part of the DICOM Worklist and that is communicated to the medical device 22.

In some embodiments, for example, in addition to the patient data being automatically populated in the patient information portion 54 and the examination data being automatically populated in the examination portion 56, corresponding presets for the examination or scan are selected and/or set, such as a preset configuration that is based on the information in the DICOM Worklist. In some embodiments, the DICOM functionality is used to allow a user to set-up or select defined or predetermined keywords for one or more DICOM tags, which are received by the medical device 22. For example, in one embodiment, DICOM tags associated with the Exam Description field 66a are searched to identify keywords that are then used to automatically select presets for the examination or scan. Thus, a user does not have to manually set up the medical device 22 for the scan. However, in some embodiments, the user may confirm or may have to confirm the settings, such as the device preset or user defined presets that were selected based on one or more keywords (or other indicators) in the DICOM examination or scan description,

In one embodiment, pre-defined keywords (e.g., alphanumeric data) are identified for a Requested Procedure Description DICOM tag, which includes in some embodiments, DICOM tag (0032,1060) received by the medical device 22 as a result of the DICOM Worklist query. Using the DICOM tags, and in particular, searching the DICOM tags for keywords, a determination may be made to the type of examination or scan to be performed, which is then correlated to a preset configuration, such that the medical device 22 is automatically configured with presets to perform the examination or scan. In some embodiments, for example, a lookup table 80 as shown in FIG. 3 may be stored in the memory of the medical device 22 that correlates keywords with one or more preset configurations. For example, the table 80 includes a column 82 of predefined keywords (or other indicators) and the preset configuration is in the corresponding row in a column 84.

It should be noted that more than one keyword or a combination of keywords (in defined or arbitrary order) may be correlated to each of the preset configurations. Additionally, as illustrated in FIG. 3, the preset configurations may be machine or device presets (U/S_{N}) that may be defined by the manufacturer or medical facility or may be user presets (User_{N}) that are defined by a user. Each of the presets corresponds to a plurality of settings for the medical device 22 as described herein. The keywords may be based on known medical terms historically used or a list may be provided when inputting the description to identify different terms. In general, the keywords are a predefined list, which may be changed from time to time.

For example if the keyword VAS-LEV (which may also be referred to as a description value) is received, the medical device 22 is configured by loading the preset for a Lower Extremity Venus Vascular Exam automatically. It should be noted that a user may be requested to confirm the preset before beginning scanning in some embodiments. It further should be noted that a user in some embodiments may set up a plurality of unique codes, values or keywords (e.g., 80 unique codes in addition to 45 standard keywords) to identify one or more preset configurations.

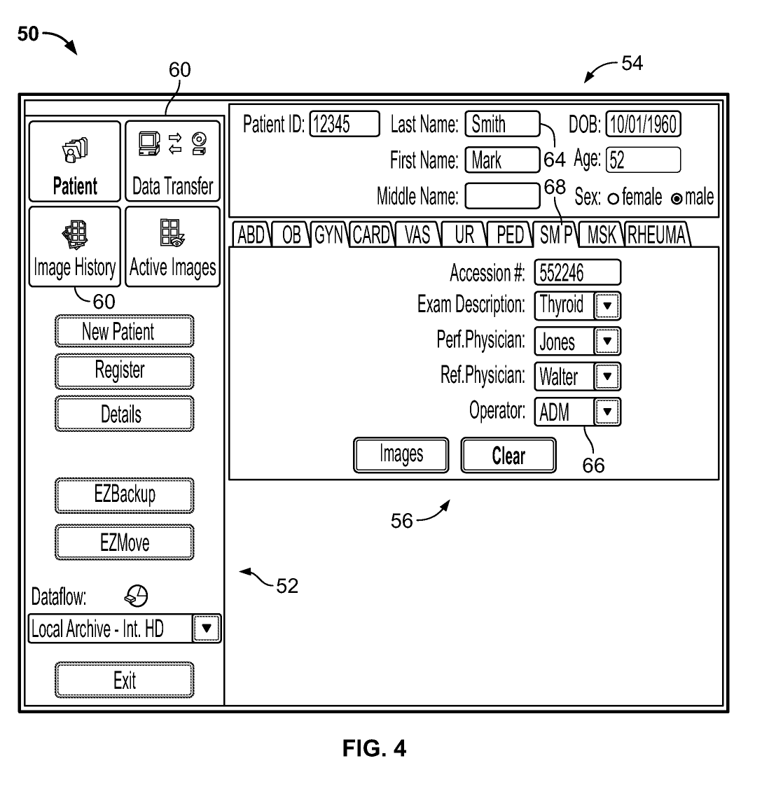

Thus, as can be seen in FIG. 4, the fields 64 and 66 in the patient information portion 54 and examination portion 56, respectively, are automatically populated with patient and examination or scan information based on, for example, a DICOM Worklist query. It should be noted that the DICOM Worklist query may be formatted using any suitable DICOM process to allow searching of DICOM Worklist data. As can be seen, in addition to populating the fields in the examination description portion 56, including the Exam Description field (a Thyroid exam in this example), the appropriate tab 68 is selected (a SMP tab in this example) for the examination or scan to be performed. Additionally, a preset configuration is selected based on the examination or scan description using one or more identified codes, values or keywords in one or more DICOM tags as described in more detail herein.

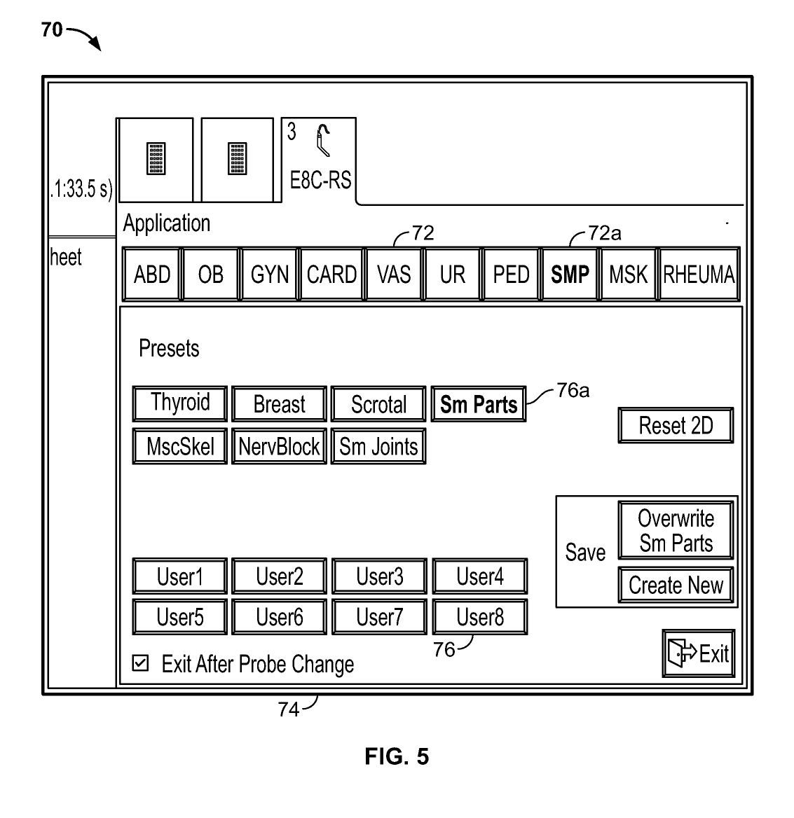

FIG. 5 is an example of a configuration screen 70 that is automatically configured with presets in accordance with various embodiments. The configuration screen 70 includes a plurality of selectable elements 72 that correspond to a plurality of examinations or scans that can be performed by the medical device 22. As can be seen, in this embodiment, the SMP scan is automatically selected corresponding to the examination description and following with the example shown in FIG. 4. A plurality of preset buttons 76 (including standard presets and user presets) are also displayed in a preset portion 74 of the configuration screen 70. In this embodiment, based on the identified examination or scan, the small parts (Sm Parts) preset is automatically selected which configures and sets up the medical device 22 (e.g., sets up scanning or acquisition parameters) for performing the examination or scan.

Thus, in various embodiments, one or more codes, values or keywords in one or more DICOM tags received by the medical device 22 are correlated with a preset for configuring the medical device 22.

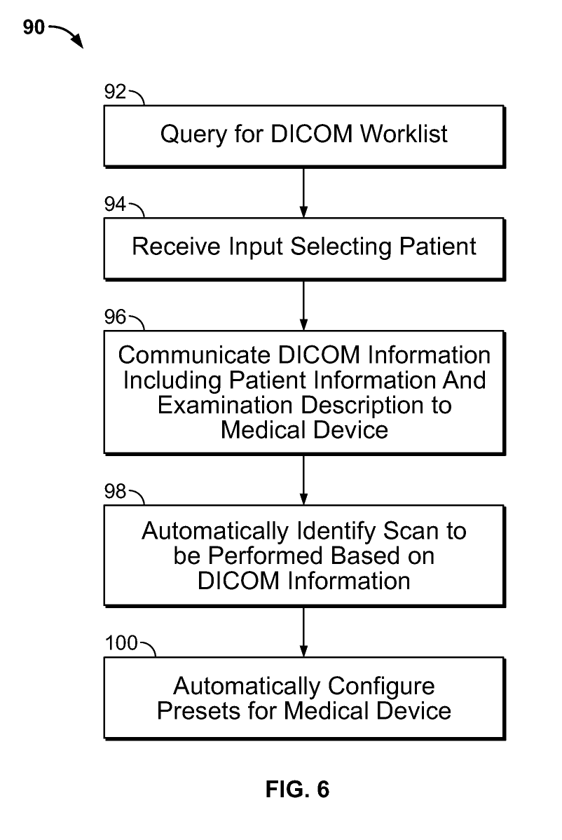

Various embodiments provide a method 90 as shown in FIG. 6 for configuring a medical device, which in one embodiment is a medical ultrasound imaging system. The method 90 includes performing a query for a DICOM Worklist at 92. For example, a user at a medical device may enter one or more search terms that queries a DICOM Worklist broker, which searches DICOM Worklists and returns results as described in more detail herein.

The method also includes receiving an input selecting a patient at 94, such as from the DICOM Worklist results. Then, based on the selection of the patient, DICOM information is communicated to the medical device at 96. For example, as described in more detail herein, DICOM Worklist information is transmitted to the medical device, which in various embodiments is patient information and examination or scan information. The patient information generally includes information that allows identification of the patient and may include different types of demographic information. The examination or scan information includes description or study information for an examination or scan to be performed. The DICOM Worklist information is transmitted using a DICOM standard.

Using the DICOM information, an examination or scan to be performed is automatically identified at 98. For example, at a user interface of the medical device, the patient information and examination or scan information is used to populate different data fields. Additionally, a user does not have to visually determine from the displayed data fields the examination or scan to be performed and then configure the medical device, for example, presets of the medical device to set up the device to perform the examination or scan. Instead, the examination or scan to be performed is determined from the DICOM information, such as based on identified codes, values or keywords, which in one embodiment is within one or more DICOM tags describing the examination or scan to be performed.

The method 90 also includes automatically configuring presets for the medical device at 100. For example, as described herein, information within the DICOM tags may be correlated to an examination or scan to be performed and the medical device is automatically configured with one or more presets for the examination or scan. Thus, a user does not have to also select the presets or settings.

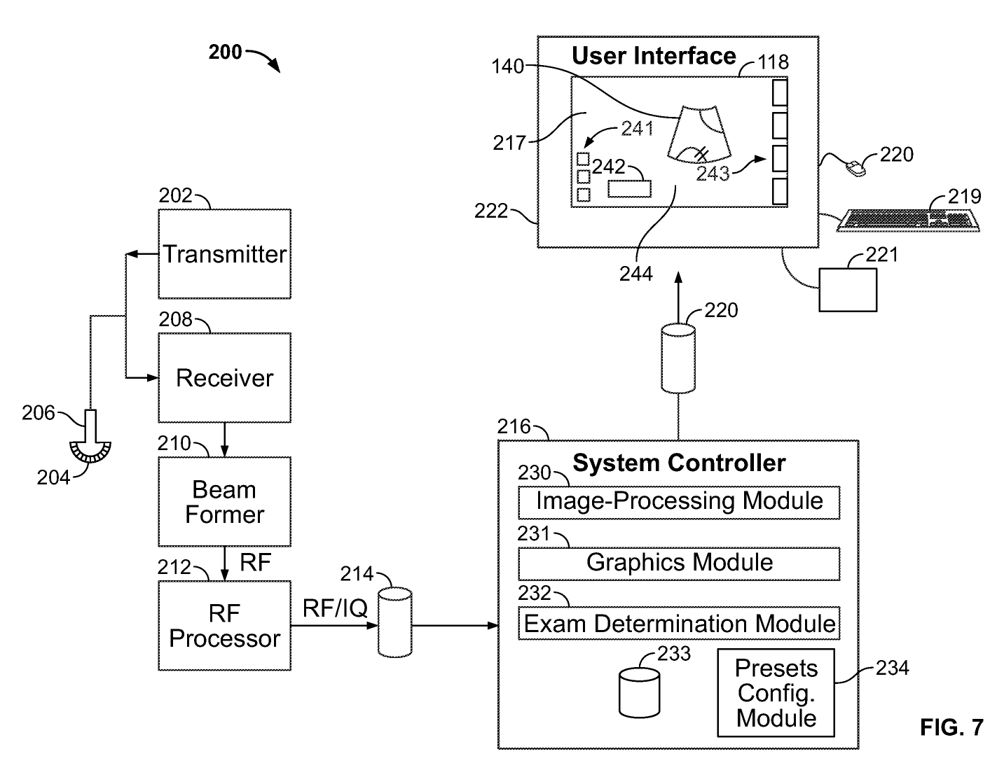

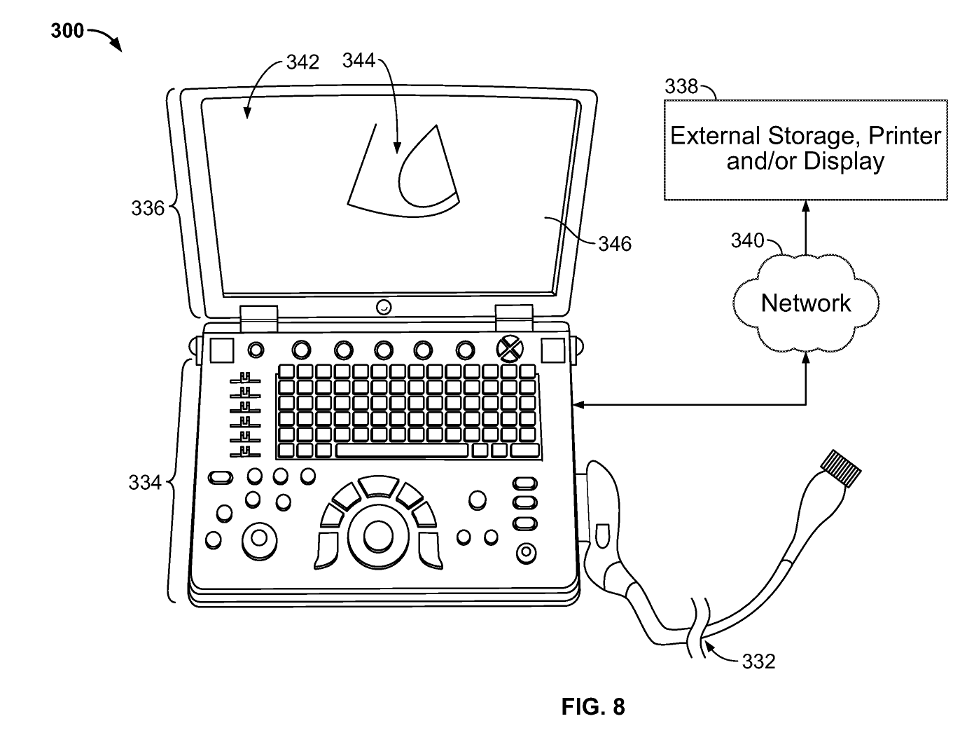

FIG. 7 illustrates a block diagram of an ultrasound system 200 according to one embodiment. The ultrasound system 200 may be a unitary apparatus such that the elements and components of the system 200 may be carried or moved with each other. The ultrasound systems 300, 350, 400 shown in FIGS. 8, 9, and 10, respectively, illustrate examples of such systems. However, in other embodiments, at least one of the system components and elements described herein may be located remotely with respect to other components and elements. For example, one or more of the described modules may operate in a data server that has a distinct and remote location with respect to an ultrasound probe and the user interface.

In the illustrated embodiment, the ultrasound system 200 includes a transmitter 202 that drives an array of elements 204, for example, piezoelectric crystals, within a diagnostic ultrasound probe 206 (or transducer) to emit pulsed ultrasonic signals into a body or volume (not shown) of a subject. The elements 204 and the probe 206 may have a variety of geometries. The ultrasonic signals are back-scattered from structures in the body, for example, blood cells or muscular tissue, to produce echoes that return to the elements 204. The echoes are received by a receiver 208. The received echoes are provided to a beamformer 210 that performs beamforming and outputs an RF signal. The RF signal is then provided to an RF processor 212 that processes the RF signal. Alternatively, the RF processor 212 may include a complex demodulator (not shown) that demodulates the RF signal to form IQ data pairs representative of the echo signals. The RF or IQ signal data may then be provided directly to a memory 214 for storage (for example, temporary storage). In the illustrated embodiment, the probe 206 is only configured for imaging. In other embodiments, the probe 206 may also be configured to provide therapy through, for example, high-intensity focused ultrasound (HIFU).

The ultrasound system 200 also includes a system controller 116 that includes a plurality of modules. The system controller 216 is configured to control operation of the ultrasound system 200. For example, the system controller 216 may include an image-processing module 230 that receives the ultrasound signals (e.g., RF signal data or IQ data pairs) and processes the ultrasound signals to generate frames of ultrasound information (e.g., ultrasound images) for displaying to the operator. The image-processing module 230 may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D), three-dimensional (3D) or four-dimensional (4D).

Acquired ultrasound information may be processed in real-time during an imaging session (or scanning session) as the echo signals are received. Additionally or alternatively, the ultrasound information may be stored temporarily in the memory 214 during an imaging session and processed in less than real-time in a live or off-line operation. An image memory 220 is included for storing processed frames of acquired ultrasound information that are not scheduled to be displayed immediately. The image memory 220 may comprise any known data storage medium, for example, a permanent storage medium, removable storage medium, and the like.

In operation, the ultrasound system 200 acquires data, for example, volumetric data sets by various techniques (e.g., 3D scanning, real-time 3D imaging, volume scanning, 2D scanning with transducers having positioning sensors, freehand scanning using a voxel correlation technique, scanning using 2D or matrix array transducers, and the like). Ultrasound images are displayed to the operator or user of the ultrasound system 200 on the display device 218.

The system controller 216 is operably connected to a user interface 222 that enables an operator to control at least some of the operations of the image-processing module 230 and to view patient and examination or scanning information. The user interface 222 may include hardware, firmware, software, or a combination thereof that enables an individual (e.g., an operator) to directly or indirectly control operation of the ultrasound system 200 and the various components thereof. As shown, the user interface 222 includes a display device 218 having a display area 217. In some embodiments, the user interface 222 may also include one or more input devices, such as a physical keyboard 219, mouse 220, and/or touchpad 221. In one embodiment, the display device 218 is a touch-sensitive display (e.g., touchscreen) that can detect a presence of a touch from the operator on the display area 217 and can also identify a location of the touch in the display area 217. The touch may be applied by, for example, at least one of an individual's hand, glove, stylus, or the like. As such, the touch-sensitive display may also be characterized as an input device that is configured to receive inputs from the operator. The display device 218 also communicates information to the operator by displaying the information to the operator. The display device 218 and/or the user interface 222 may also be configured to receive audible inputs or commands. The display device 118 is configured to present information to the operator during the imaging session. The information presented may include ultrasound images, graphical elements, user-selectable elements, and other information (e.g., administrative information, personal information of the patient, and the like).

The system controller 216 also includes a graphics module 231, an exam detection module 232, a database 233 and a presets configuration module 234. The image-processing module 230, the graphics module 231, the content detection module 232 and presets configuration module 234 coordinate with one another to present information to the operator during the imaging session and set up or configure the ultrasound system 200 as described in more detail herein. For example, the image-processing module 230 may be configured to generate for display an acquired image 240 on the display device 218, and the graphics module 231 may be configured to display designated graphics along with the ultrasound images. The graphics may include icons 241, data fields 242, user-selectable elements 243, and the like.

The exam detection module 232 is configured to automatically detect an examination or scan to be performed and which is then used by the presets configuration module 234 to automatically select a presets configuration as described in more detail herein. For example, in some embodiments, based on the detected examination or scan to be performed, a table within the database 133 is accessed to correlate the detected examination or scan, which may be based on information within one or more received DICOM tags, to one or more preset(s) configuration(s). It should be noted that in various embodiments, the acquisition parameters are selected based on the presets without user input. It also should be noted that the operator may be enabled to activate one of the user-selectable elements 243 to, for example, make adjustments to the displayed image 240 or to select different images (or to perform DICOM Worklist searches or confirm the automatically configured presets).

FIG. 8 illustrates a portable ultrasound system 300 having an ultrasound transducer 332 that may be configured to acquire ultrasonic data. For example, the ultrasound transducer 332 may have a 2D array of acoustic elements. A user interface 334 (that may also include an integrated display 336) is provided to receive commands from an operator. As used herein, “portable” includes a handheld or hand-carried device that may be carried in a person's hand, pocket, briefcase-sized case, or backpack. For example, the ultrasound system 300 may be a hand-carried device having a size of a typical laptop computer. The integrated display 336 (e.g., an internal display) is configured to display, for example, one or more medical images. The ultrasonic data may be sent to an external device 338 via a wired or wireless network 340 (or direct connection, for example, via a serial or parallel cable or USB port). In some embodiments, the external device 338 may be a computing system (e.g., computer, server, and the like).

The display 336 may be configured to show an imaging screen and a transparent object or region of interest (or surrounding region) such as described above. For instance, the display 336 includes a display area 342 that is configured to display an acquired ultrasound image 344. In the illustrated embodiment, the visualization of a device 344 is enhanced by adaptive opacity changes to allow viewing of the entire device 344 on both sides of an opening 348 (including behind tissue that would otherwise cause blurring).

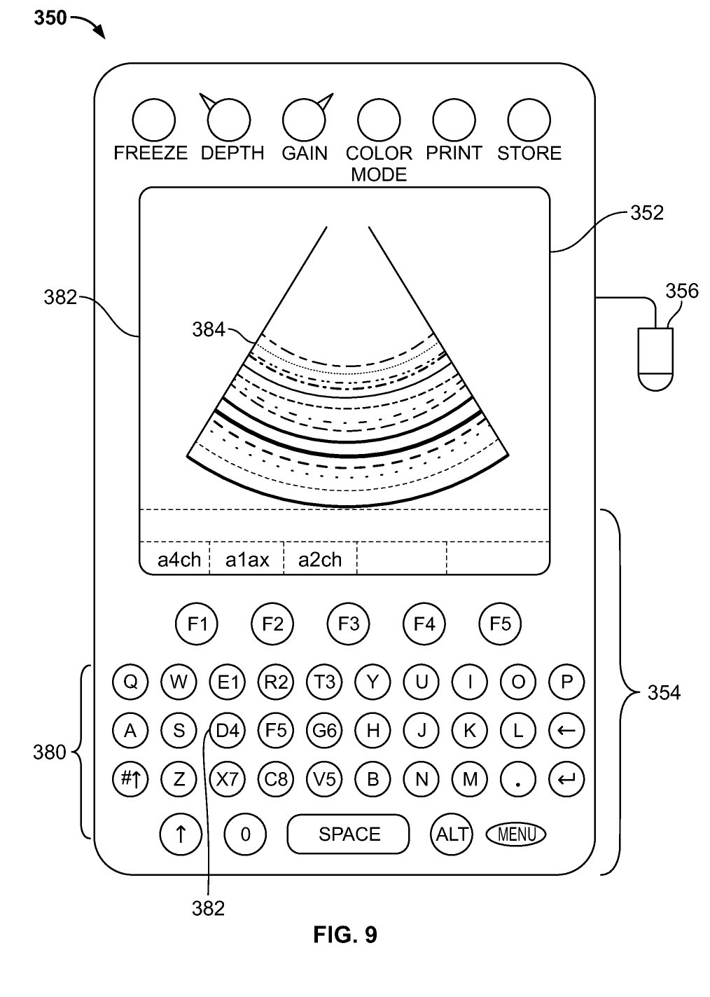

FIG. 9 illustrates a portable hand-carried or pocket-sized ultrasound imaging system 350 wherein a display 352 and a user interface 354 form a single unit. By way of example, the pocket-sized ultrasound imaging system 350 may be a personal communication device, such as a smartphone or tablet. By way of example, the personal communication device may be dimensioned to be less than 3 inches wide, less than 4 inches in length, and less than 0.5 inches in depth, less than 8 ounces in weight. The portable ultrasound imaging system 350 generally includes the display 352, user interface 354, which may or may not include a keyboard-type interface and an input/output (I/O) port for connection to a scanning device, for example, and an ultrasound transducer 356. The display 352 may be, for example, a 320×320 pixel color LCD display (on which a medical image 390 may be displayed). A typewriter-like keyboard 380 of buttons 382 may optionally be included in the user interface 354. In other embodiments, the display 352 may be larger and a virtual keyboard may be shown.

In FIG. 9, the display 352 includes a display area 382 that includes an acquired image 384 and an identified device 386, which in this example has a changed appearance (e.g., a model or mesh overlay).

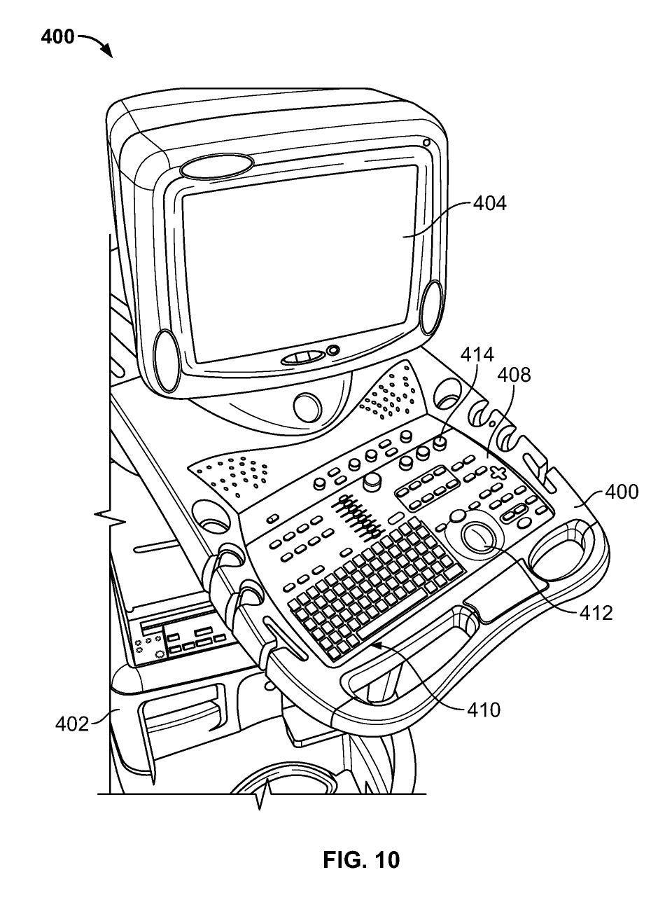

FIG. 10 illustrates an ultrasound imaging system 400 provided on a movable base 402. The portable ultrasound imaging system 400 may also be referred to as a cart-based system. A display 404 and user interface 406 are provided and it should be understood that the display 404 may be separate or separable from the user interface 406. The user interface 406 may optionally be a touchscreen, allowing the operator to select options by touching displayed graphics, icons, and/or the like.

The user interface 406 also includes control buttons 408 that may be used to control the portable ultrasound imaging system 400 as desired or needed, and/or as typically provided. The user interface 406 provides multiple interface options that the user may physically manipulate to interact with ultrasound data and other data that may be displayed, as well as to input information and set and change scanning parameters and viewing angles, etc. For example, a keyboard 410, trackball 412 and/or multi-function controls 414 may be provided.

It should be noted that although one or more embodiments may be described in connection with an ultrasound system, the embodiments described herein are not limited to ultrasound systems. In particular, one or more embodiments may be implemented in connection with different types of medical imaging systems. Examples of such medical imaging systems include a magnetic resonance imaging (MRI) system, computed tomography (CT) system, positron emission tomography (PET) system, a PET/CT system, and single photon emission computed tomography (SPECT) system. In such embodiments, the acquired images may be MRI images, CT images, PET images, PET/CT images, and SPECT images.

As used herein, the term “computing system” or “system controller” may include any processor-based or microprocessor-based systems including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computing system” or “system controller.”

Sets of instructions may include various commands that instruct the computing system or system controller as a processing machine to perform specific operations such as the methods and processes described herein. The set of instructions may be in the form of a software program or module. The software may be in various forms such as system software or application software. In some embodiments, a tangible and non-transitory computer readable medium is provided. Further, the software may be in the form of a collection of separate programs, a program module (or module) within a larger program, or a portion of a program module. The software also may include modular programming in the form of object-oriented programming. The processing of input data by the processing machine may be in response to user commands, or in response to results of previous processing, or in response to a request made by another processing machine. The program is configured to run on both 32-bit and 64-bit operating systems. A 32-bit operating system like Windows XP™ can only use up to 3 GB bytes of memory, while a 64-bit operating system like Window's Vista™ can use as many as 16 exabytes (16 billion GB). In some embodiments, the program is configured to be executed on a Linux-based system.

As used herein, the terms “software” and “firmware” are interchangeable, and include any computer program stored in memory for execution by a computing system, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein are intended to define the parameters of various embodiments, they are by no means limiting and are only example embodiments. Many other embodiments will be apparent to those of skill in the art upon reviewing the above description. The scope of the present application should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A medical system comprising:
a computer configured to access a plurality of stored Digital Imaging and Communications in Medicine (DICOM) worklists and transmit DICOM worklist information; and
a medical device configured to receive the DICOM worklist information including examination or scan information for the medical device and determine a preset configuration for the medical device corresponding to an examination or scan using the examination or scan information, the medical device further configured to automatically select the preset configuration to set one or more acquisition parameters for the examination or scan.

2. The medical system of claim 1, wherein the examination or scan information is in a defined DICOM tag and the medical device is configured to identify a code, value or keyword within the DICOM tag corresponding to the examination or scan information.

3. The medical system of claim 2, wherein the medical device is configured to correlate the identified code, value or keyword to the preset configuration.

4. The medical system of claim 2, wherein the DICOM tag is a Requested Procedure Description (0032,1060) tag.

5. The medical system of claim 1, wherein the medical device is an ultrasound system and the preset configuration comprises one or more probe acquisition parameters.

6. The medical system of claim 1, wherein the preset configuration is selected from a plurality of predefined or user defined settings.

7. The medical system of claim 1, wherein the medical device is configured to receive user confirmation of the automatically selected preset configuration.

8. The medical system of claim 1, wherein the computer comprises a DICOM Worklist broker.

9. An ultrasound imaging system comprising:
a user interface having a display configured to display patient and examination information;
an examination determination module configured to determine, from received Digital Imaging and Communications in Medicine (DICOM) worklist information including examination or scan information, a preset configuration corresponding to an examination or scan using the examination or scan information; and
a presets configuration module configured to automatically select the preset configuration to set one or more acquisition parameters for the examination or scan.

10. The ultrasound imaging system of claim 9, wherein the examination or scan information is in a defined DICOM tag and the examination determination module is configured to identify a code, value or keyword within the DICOM tag corresponding to the examination or scan information.

11. The ultrasound imaging system of claim 10, wherein the examination determination module is configured to correlate the identified code, value or keyword to the preset configuration.

12. The ultrasound imaging system of claim 10, wherein the DICOM tag is a Requested Procedure Description (0032,1060) tag.

13. The ultrasound imaging system of claim 9, wherein the preset configuration comprises one or more probe acquisition parameters.

14. The ultrasound imaging system of claim 9, wherein the preset configuration is selected from a plurality of predefined or user defined settings.

15. The ultrasound imaging system of claim 9, wherein the user interface is configured to receive user confirmation of the automatically selected preset configuration.

16. The ultrasound imaging system of claim 9, wherein a DICOM Worklist broker transmits the DICOM worklist information.

17. A method for configuring a medical device, the method comprising:
determining, from received Digital Imaging and Communications in Medicine (DICOM) worklist information including examination or scan information, a preset configuration corresponding to an examination or scan using the examination or scan information; and
automatically selecting the preset configuration to set one or more acquisition parameters for the examination or scan.

18. The method of claim 17, wherein the examination or scan information is in a defined DICOM tag and further comprising identifying a code, value or keyword within the DICOM tag corresponding to the examination or scan information and correlating the identified code, value or keyword to the preset configuration.

19. The method of claim 18, wherein the DICOM tag is a Requested Procedure Description (0032,1060) tag.

20. The method of claim 17, wherein the preset configuration comprises one or more probe acquisition parameters and is selected from a plurality of predefined or user defined settings.
